(19) United States
(12) Patent Application Publication
(10) Pub. No.: US 2025/0345117 A1
(43) Pub. Date: Nov. 13, 2025

(54) DESIGN METHOD OF ANATOMICAL PLATE FOR TREATING DISTAL FEMORAL FRACTURE AND ANATOMICAL PLATE

(71) Applicants: THE FOURTH MEDICAL CENTER OF PLA GENERAL HOSPITAL, Beijing (CN); Beijing Libeier Bio-engineering Institute Co., Ltd., Beijing (CN)

(72) Inventors: Wei Zhang, Beijing (CN); Bowen Yuan, Beijing (CN); Meng Li, Beijing (CN); Jiantao Li, Beijing (CN); Jian Li, Beijing (CN); Zicheng Zhang, Beijing (CN); Mengmeng Hu, Beijing (CN); Dong Zhang, Beijing (CN); Ting Li, Beijing (CN); Mingli Liu, Beijing (CN); Xiaomeng Ren, Beijing (CN); Yi Gao, Beijing (CN); Longbo Du, Beijing (CN); Rui Ma, Beijing (CN); Yuchen Guo, Beijing (CN)

(21) Appl. No.: 19/070,483

(22) Filed: Mar. 4, 2025

(30) Foreign Application Priority Data
May 7, 2024 (CN) 202410553758.1

(51) Int. Cl. A61B 34/10; A61B 17/74

(52) U.S. Cl. CPC A61B 34/10; A61B 17/742; A61B 2034/102

(57) ABSTRACT

A design method of an anatomical plate for treating a distal femoral fracture and an anatomical plate are provided. The design method includes: obtaining a high-precision three-dimensional model of a fractured bone of a patient; simulating intraoperative reduction to obtain a post-reduction bone model; establishing a finite element model and performing finite element analysis to determine a force condition of the post-reduction bone model; extracting point arrays of a cross section and a coronal plane from a medial femoral condyle part and a diaphysis part of a distal femur of the post-reduction bone model, connecting the point arrays of the cross section and the coronal plane to obtain a connection curve, and obtaining a solid body design of a plate body; adjusting a thickness and a shape of the plate body according to the force condition and an implantation condition of the post-reduction bone model to obtain an anatomical plate.

### CROSS-REFERENCE TO RELATED APPLICATION

This application claims priority of Chinese Patent Application No. 202410553758.1, filed on May 7, 2024, the entire contents of which are incorporated herein by reference.

### TECHNICAL FIELD

The present disclosure relates to the technical field of medical apparatus and instruments, and in particular, to a design method of an anatomical plate for treating a distal femoral fracture and an anatomical plate.

### BACKGROUND

Distal femoral fractures account for 3-6% of all femoral fractures, which are the second major femoral fractures ranking only second to proximal femoral fractures. These fractures are common in young patients with high-energy trauma or elderly patients with low-energy trauma. However, about 15-20% of distal femoral fractures might cause complications such as bone ununion. The increase of the incidence rate of these fractures brings increasingly great social and economic burdens for the society. Especially for elderly patients, their bone quality is relatively poor, and stress shielding caused by systemic diseases, osteoporosis, or protheses may easily result in prosthesis loosening, prosthesis fracture, bone ununion, or malunion, etc. It needs to be noted that the death rate of elderly patients with the distal femoral fractures in the first year after surgeries may be up to 30%, which might be related with insufficient initial stability and lack of weight training after surgeries.

The biomechanical advantages of a two-plate fixation way in treating the distal femoral fractures have been verified. The two-plate fixation way can stabilizer two props at the distal femur and provide stronger fixation for comminuted femoral supracondylar fractures, low fractures around prostheses, and bone ununion. The problem that eccentric fixation of a single lateral plate affects porosis can be solved to a certain extent. However, it is reported in documents that the two-plate fixation way is not superior to the traditional lateral locking plate in terms of healing rate, functional outcome, and complication, which is mainly due to lack of an effective medial support plate.

Medial plates are usually used in two ways. One way is to use a medial plate in combination with a lateral plate, and this way is suitable for a case in which fractures occur in medial and lateral femoral condyles, and may have a great influence on the force condition of a patient. The other way is to use a medial plate solely, and this way is suitable for a case in which only a medial condyle fracture occurs. In this case, the medial plate needs to bear a force, and a high requirement is imposed on plate strength. However, the two ways currently have no suitable medial fixation plate systems matching the anatomic forms of patients.

Therefore, if a plate does not match the anatomic form or the fracture form of a patient, it is difficult to provide enough stability by using a traditional internal fixation system (e.g., a traditional fixation way with a plate and a screw), leading to unstable fixation and affecting the healing process of the fracture.

### SUMMARY

The present disclosure provides a design method of an anatomical plate for treating a distal femoral fracture and an anatomical plate to solve the problem that unstable fixation of a traditional internal fixation system affects the healing process of a fracture in the related art.

According to one aspect of the present disclosure, there is provided a design method of an anatomical plate for treating a distal femoral fracture. The design method of an anatomical plate for treating a distal femoral fracture includes: obtaining a high-precision three-dimensional model of a fractured bone of a patient; simulating intraoperative reduction on the high-precision three-dimensional model to obtain a post-reduction bone model; establishing a finite element model according to the post-reduction bone model and performing finite element analysis to determine a force condition of the post-reduction bone model; extracting point arrays of a cross section and a coronal plane from a medial femoral condyle part and a diaphysis part of a distal femur of the post-reduction bone model, connecting the point arrays of the cross section and the coronal plane to obtain a connection curve, and obtaining a solid body design of a plate body according to the connection curve; and adjusting a thickness and a shape of the plate body according to the force condition and an implantation condition of the post-reduction bone model to obtain an anatomical plate.

Further, the design method of an anatomical plate for treating a distal femoral fracture may further include: combining the post-reduction bone model with the anatomical plate to obtain a combined model; performing finite element analysis on the combined model; if the combined model meets a force requirement, determining that the design of the anatomical plate is completed; and if the combined model does not meet the force requirement, adjusting the thickness and the shape of the plate body according to the force condition and the implantation condition of the post-reduction bone model to obtain the anatomical plate until the combined model meets the force requirement.

Further, the adjusting a thickness and a shape of the plate body according to the force condition and an implantation condition of the post-reduction bone model to obtain an anatomical plate may include: if the anatomical plate is a medial plate and a non-force-bearing plate, determining that the anatomical plate needs to be used in combination with a lateral plate, and designing a thickness of a distal end of the anatomical plate to be 3 mm to 5 mm and a thickness of a main body of the anatomical plate to be 4 mm to 6 mm; and if the anatomical plate is a medial plate and a force-bearing plate, adjusting the thickness of the distal end of the anatomical plate according to a condition of soft tissue of the patient, and adjusting the thickness of the main body of the anatomical plate according to the force condition of the post-reduction bone model.

Further, the adjusting the thickness of the distal end of the anatomical plate according to a condition of soft tissue of the patient may include: if a thickness of the soft tissue of the patient is less than 20 mm, setting the thickness of the distal end of the anatomical plate to be 3 mm to 3.5 mm; if the thickness of the soft tissue of the patient is 20 mm to 30 mm, setting the thickness of the distal end of the anatomical plate to be 3.5 mm to 4.5 mm; and if the thickness of the soft tissue of the patient is greater than 30 mm, setting the thickness of the distal end of the anatomical plate to be 4.5 mm to 5 mm.

Further, the adjusting the thickness of the main body of the anatomical plate according to the force condition of the post-reduction bone model may include: if a maximum stress of the post-reduction bone model is less than 100 Mpa, setting the thickness of the main body of the anatomical plate to be 4 mm to 4.5 mm; if the maximum stress of the post-reduction bone model is 100 Mpa to 150 Mpa, setting the thickness of the main body of the anatomical plate to be 4.5 mm to 5 mm; and if the maximum stress of the post-reduction bone model is greater than 150 Mpa, setting the thickness of the main body of the anatomical plate to be 5 mm to 6 mm.

Further, the adjusting a thickness and a shape of the plate body according to the force condition and an implantation condition of the post-reduction bone model to obtain an anatomical plate may further include: designing a lower edge of the plate body to be 1 mm to 3 mm higher than a femoral condyle joint surface, and designing a width of a distal end of the plate body to be 25 mm to 35 mm; designing the distal end of the plate body to extend to a foremost edge of an inner side of the femoral condyle, and designing a main body of the plate body to extend to a front inner side of the distal femur; and designing a width of the main body of the plate body to be 14 mm to 17 mm.

Further, the adjusting a thickness and a shape of the plate body according to the force condition and an implantation condition of the post-reduction bone model to obtain an anatomical plate may further include: obtaining a soft tissue condition around the bone of the patient, and adding a bevel angle and a fillet angle to the anatomical plate in accordance with the soft tissue condition and an implantation position of the anatomical plate.

Further, the design method of an anatomical plate for treating a distal femoral fracture may further include: obtaining a position of a bone fragment and determining a position and a direction of a screw hole in accordance with the position of the bone fragment and the force condition of the post-reduction bone model.

Further, the obtaining a position of a bone fragment and determining a position and a direction of a screw hole in accordance with the position of the bone fragment and the force condition of the post-reduction bone model may include: designing a plurality of first screw holes in the distal end of the plate body, where the foremost first screw hole of the plurality of first screw holes is disposed corresponding to a lateral condyle, and the first screw hole of the plurality of first screw holes that is located in the middle of a farthest end is disposed corresponding to a medial condyle; and the other first screw holes are disposed perpendicular to a sagittal plane; designing at least two screw holes in a neck of the plate body, where the at least two screw holes are disposed perpendicular to the sagittal plane; and designing a third screw hole in the main body of the plate body, where the third screw hole is perpendicular to the plate body.

According to another aspect of the present disclosure, there is provided an anatomical plate, manufactured in accordance with the design method of an anatomical plate for treating a distal femoral fracture described above.

With the technical solutions of the present disclosure, the high-precision three-dimensional model of the fractured bone of the patient is obtained first, and then the intraoperative reduction is simulated on the high-precision three-dimensional model to obtain the post-reduction bone model. Thus, the obtained model can match the anatomic form of the patient. Then, the finite element model is established according to the post-reduction bone model and finite element analysis is performed to determine the force condition of the post-reduction bone model. The point arrays of the cross section and the coronal plane are extracted from the medial femoral condyle part and the diaphysis part of the distal femur of the post-reduction bone model, and the point arrays of the cross section and the coronal plane are connected to obtain the connection curve; and the solid body design of the plate body is obtained according to the connection curve. Finally, the thickness and the shape of the plate body are adjusted according to the force condition and the implantation condition of the post-reduction bone model to obtain the anatomical plate. By the above-mentioned design method, since the obtained model matches the anatomic form of the patient, the force condition of the post-reduction bone model is determined through the finite element analysis such that the model is fitter with the state of the patient after recovery. Moreover, the solid body design of the plate body is obtained with the point arrays of the cross section and the coronal plane and then the thickness and the shape of the plate body are adjusted according to the force condition and the implantation condition of the post-reduction bone model to obtain the anatomical plate so that the design precision of the anatomical plate can be guaranteed, making the anatomical plate match the anatomic form or the fracture form of the patient. Thus, enough stability can be provided, providing stable fixation and being conducive to the healing process of the fracture.

### BRIEF DESCRIPTION OF THE DRAWINGS

The accompany drawings constituting a part of the present disclosure provide further understanding of the present disclosure. The schematic examples of the present disclosure and description thereof are intended to be illustrative of the present disclosure and do not constitute an undue limitation of the present disclosure. In the accompanying drawings:

FIG. 1 is a flowchart of a design method of an anatomical plate for treating a distal femoral fracture provided by an embodiment of the present disclosure;

FIG. 2 is a schematic diagram of a post-reduction bone model in a design method of an anatomical plate for treating a distal femoral fracture provided by an embodiment of the present disclosure;

FIG. 3 is a schematic diagram of picking up a point array in a design method of an anatomical plate for treating a distal femoral fracture provided by an embodiment of the present disclosure;

FIG. 4 is a schematic diagram of a connection curve in a design method of an anatomical plate for treating a distal femoral fracture provided by an embodiment of the present disclosure;

FIG. 5 is a schematic diagram of a connection curve in a design method of an anatomical plate for treating a distal femoral fracture provided by an embodiment of the present disclosure;

FIG. 6 is a schematic diagram of a force on a model in a design method of an anatomical plate for treating a distal femoral fracture provided by an embodiment of the present disclosure;

FIG. 7 is a schematic diagram of a plate body in a design method of an anatomical plate for treating a distal femoral fracture provided by an embodiment of the present disclosure;

FIG. 8 is a schematic diagram of an anatomical plate in a design method of an anatomical plate for treating a distal femoral fracture provided by an embodiment of the present disclosure;

FIG. 9 is a schematic diagram of an anatomical plate in a design method of an anatomical plate for treating a distal femoral fracture provided by an embodiment of the present disclosure;

FIG. 10 is a schematic diagram of an anatomical plate in a design method of an anatomical plate for treating a distal femoral fracture provided by an embodiment of the present disclosure;

FIG. 11 is a schematic diagram of an anatomical plate in a design method of an anatomical plate for treating a distal femoral fracture provided by an embodiment of the present disclosure;

FIG. 12 is a schematic diagram of an anatomical plate in a design method of an anatomical plate for treating a distal femoral fracture provided by an embodiment of the present disclosure;

FIG. 13 is a schematic diagram of an anatomical plate in a design method of an anatomical plate for treating a distal femoral fracture provided by an embodiment of the present disclosure;

FIG. 14 is a schematic diagram of an anatomical plate in a design method of an anatomical plate for treating a distal femoral fracture provided by an embodiment of the present disclosure;

FIG. 15 is a schematic diagram of an anatomical plate in a design method of an anatomical plate for treating a distal femoral fracture provided by an embodiment of the present disclosure;

FIG. 16 is a schematic diagram of an anatomical plate matching a bone in a design method of an anatomical plate for treating a distal femoral fracture provided by an embodiment of the present disclosure; and

FIG. 17 is a schematic diagram of an anatomical plate matching a bone in a design method of an anatomical plate for treating a distal femoral fracture provided by an embodiment of the present disclosure.

### Reference numerals in the drawings are as follows:

10—post-reduction bone model; 11—plate body; 12—anatomical plate; and 13—connection curve.

### DETAILED DESCRIPTION OF THE EMBODIMENTS

The technical solutions of the embodiments of the present disclosure are clearly and completely described below with reference to the accompanying drawings. Apparently, the described embodiments are merely a part rather than all of the embodiments of the present disclosure. The following description of at least one exemplary embodiment is merely illustrative, and not intended to limit the present disclosure and application or use thereof in any way. All other embodiments derived from the embodiments of the present disclosure by a person of ordinary skill in the art without creative efforts shall fall within the protection scope of the present disclosure.

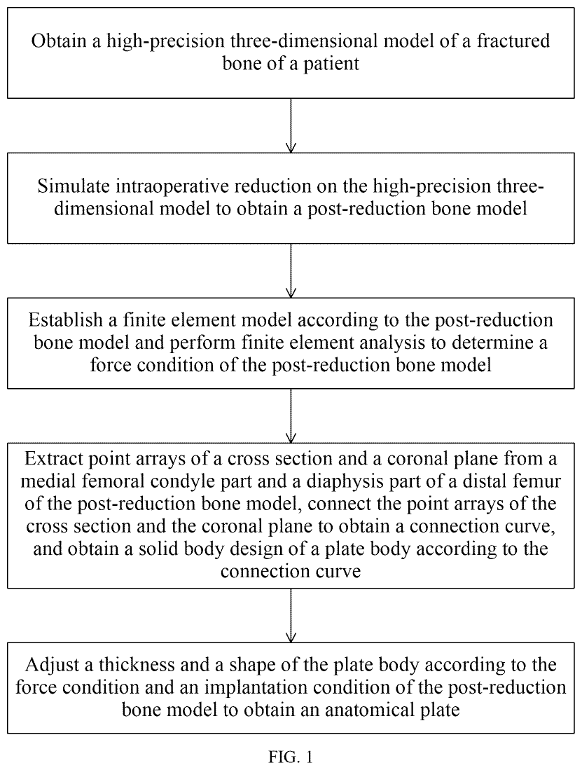

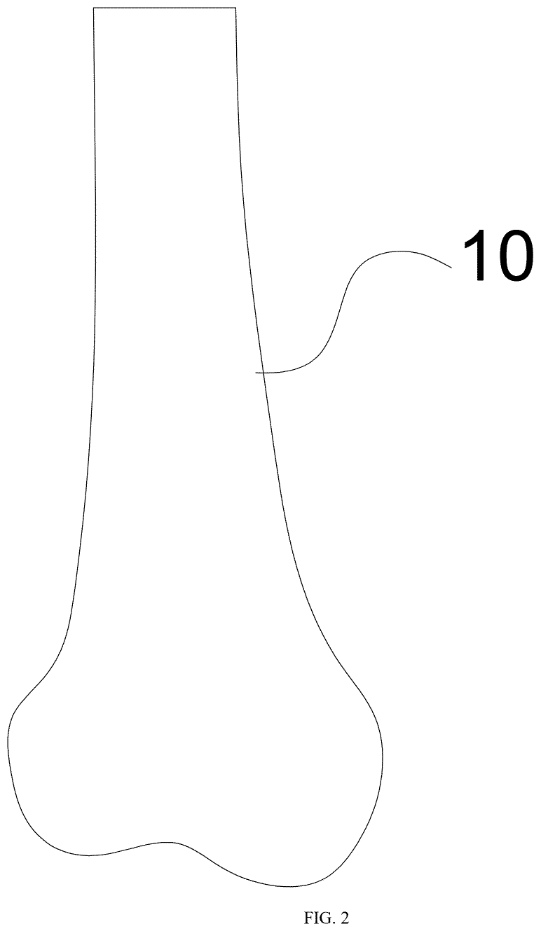

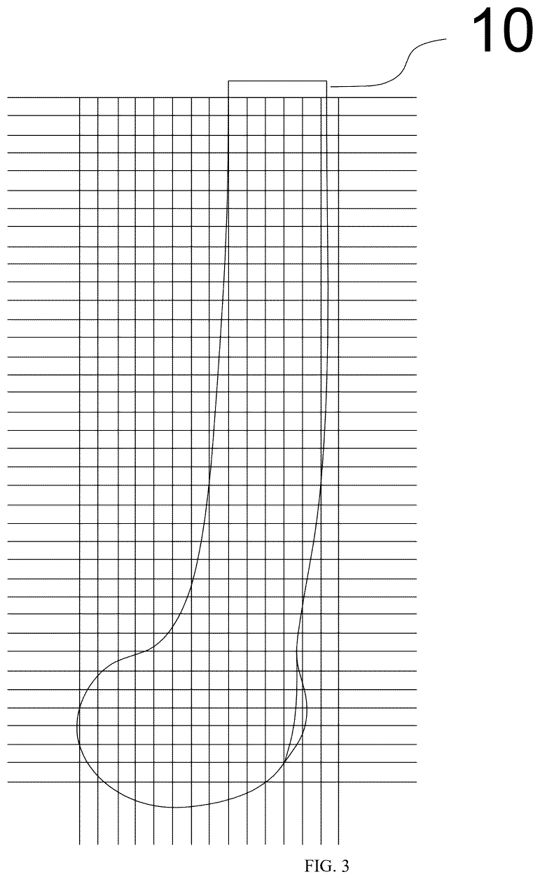

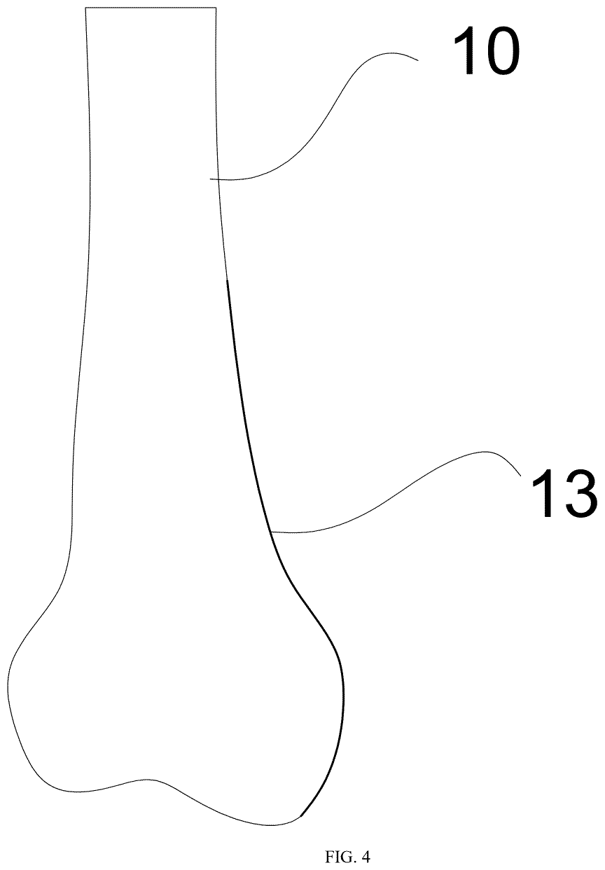

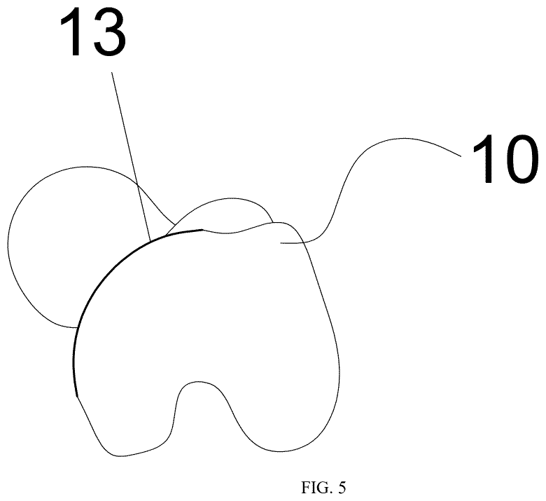

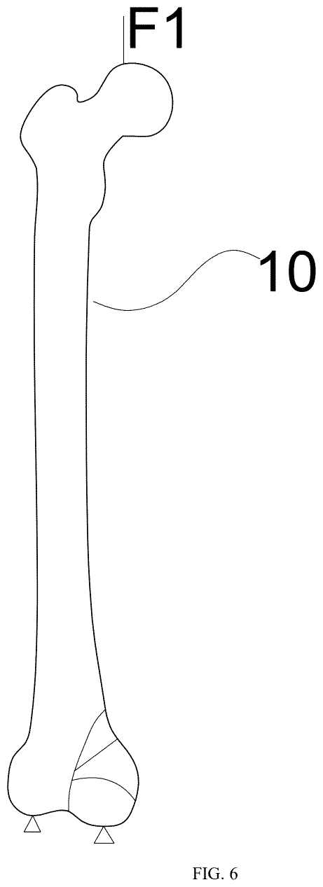

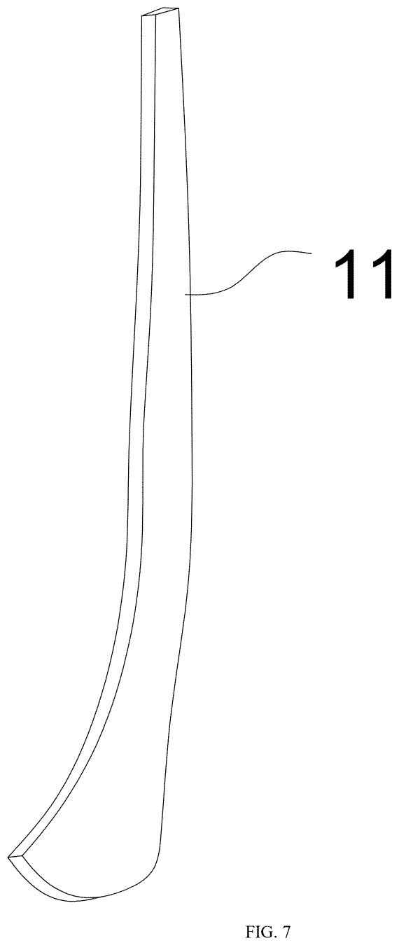

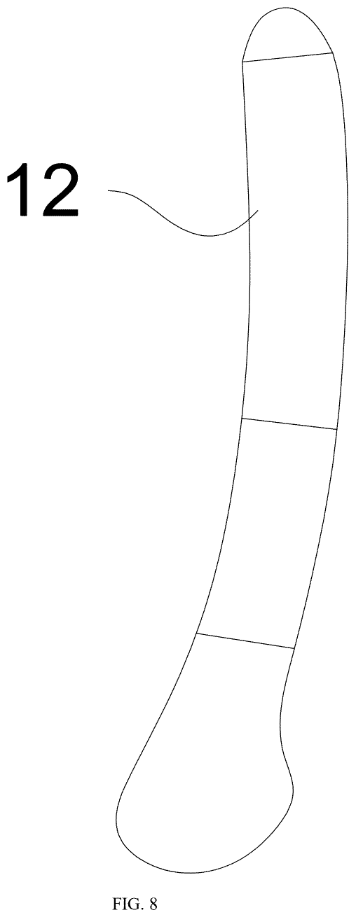

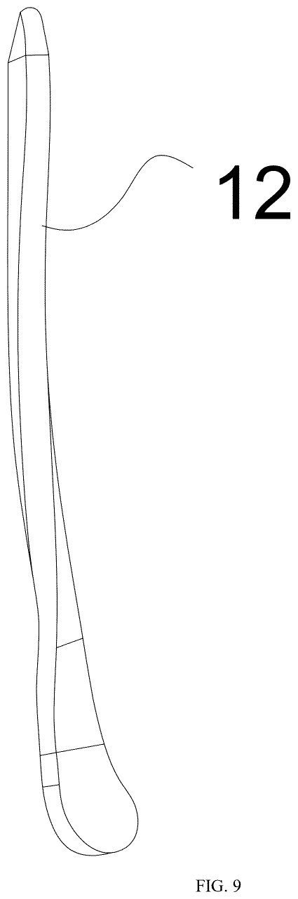

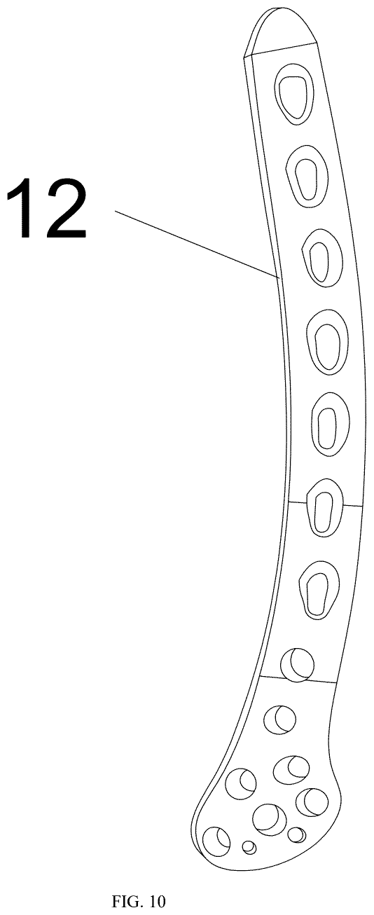

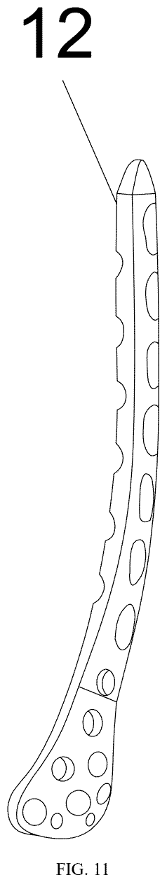

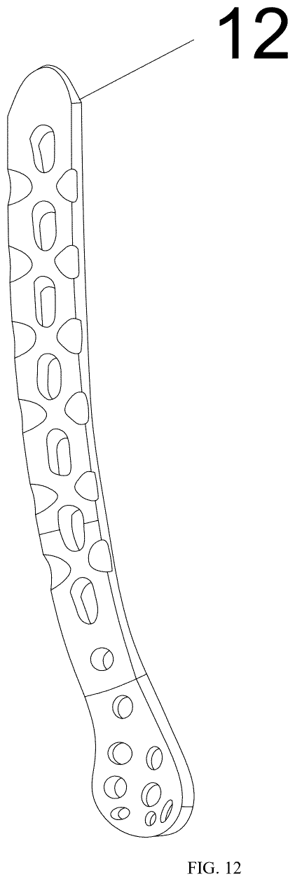

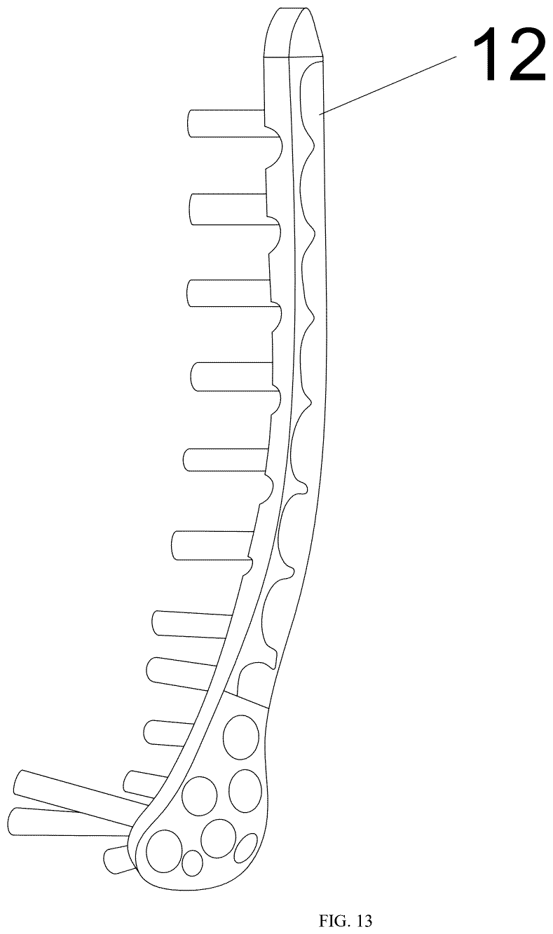

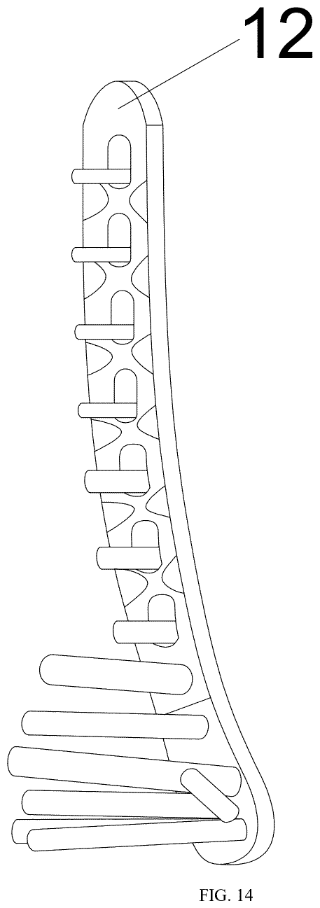

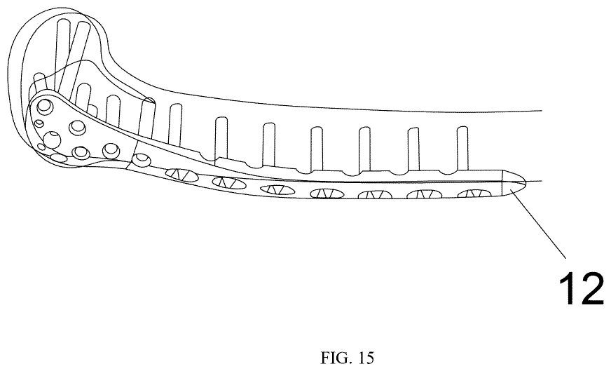

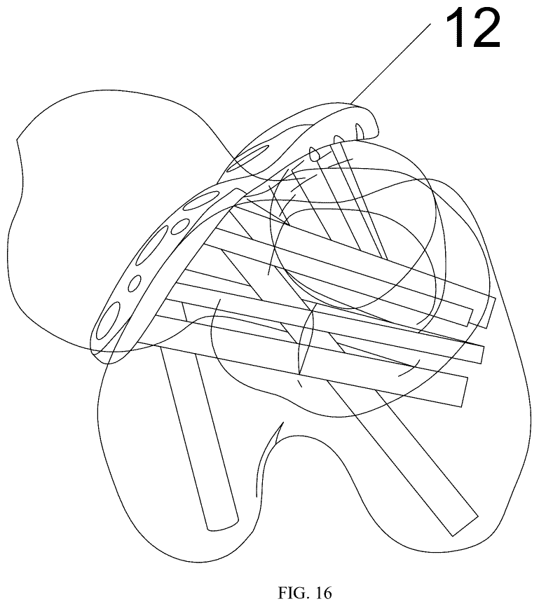

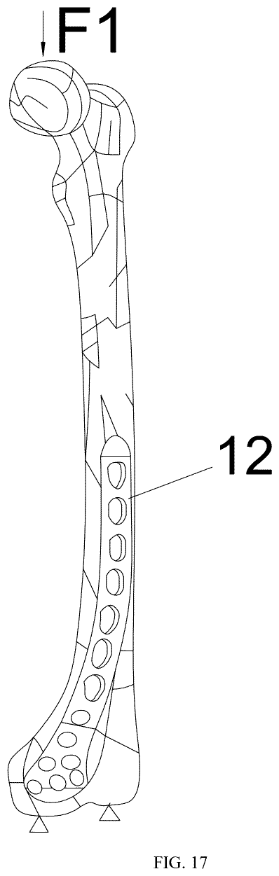

As shown in FIG. 1 to FIG. 17, an embodiment of the present disclosure provides a design method of an anatomical plate for treating a distal femoral fracture. The design method of an anatomical plate for treating a distal femoral fracture includes the following steps.

A high-precision three-dimensional model of a fractured bone of a patient is obtained.

Intraoperative reduction is simulated on the high-precision three-dimensional model to obtain a post-reduction bone model 10, for ensuring the closure of a fracture line while taking the fracture stability and functionality requirements under different postoperative load conditions into account.

A finite element model is established according the post-reduction bone model 10 and finite element analysis is performed to determine a force condition of the post-reduction bone model 10. Specifically, a force concentration point of the post-reduction bone model 10 is determined. With an internal fixation design, an internal fixation strength requirement is met.

Point arrays of a cross section and a coronal plane are extracted from a medial femoral condyle part and a diaphysis part of a distal femur of the post-reduction bone model 10, and the point arrays of the cross section and the coronal plane are connected to obtain a connection curve 13; and optimization is made according to an anatomical condition to ensure that a shape and a structure of an entity part meet more accurate treatment requirements. a solid body design of a plate body 11 is obtained according to the connection curve 13.

A thickness and a shape of the plate body 11 are adjusted according to the force condition and an implantation condition of the post-reduction bone model 10 to obtain an optimal solid body design, thereby obtaining an anatomical plate 12.

By the design method of an anatomical plate for treating a distal femoral fracture, since the obtained model matches the anatomic form of the patient, the force condition of the post-reduction bone model 10 is determined through the finite element analysis such that the model is fitter with the state of the patient after recovery. Moreover, the solid body design of the plate body 11 is obtained with the point arrays of the cross section and the coronal plane and then the thickness and the shape of the plate body 11 are adjusted according to the force condition and the implantation condition of the post-reduction bone model 10 to obtain the anatomical plate 12 so that the design precision of the anatomical plate 12 can be guaranteed, making the anatomical plate 12 match the anatomic form or the fracture form of the patient. Thus, enough stability can be provided, providing stable fixation and being conducive to the healing process of the fracture.

In the related art, a complex fracture repair surgery might increase the risk of postoperative infection, especially when there are many bone fragments and the soft tissue injury is obvious. Moreover, the traditional internal fixation system might require a long-time rehabilitation period, and the function recovery might be limited, thus affecting the living quality of the patient. Since the surgical injury range is expanded, the surrounding tissue might be secondarily injured, causing extra trouble and complications. The design method of an anatomical plate for treating a distal femoral fracture provided in this embodiment can enable the anatomical plate 12 to match the anatomic form or the fracture form of the patient, simplify the fracture repair surgery, reduce the risk of postoperative infection, shorten the function recovery time, and avoid or reduce secondary injuries.

In this embodiment, a digital imaging and communications in medicine (DICOM) data set of the patient is obtained by imaging techniques such as high-precision computed tomography (CT), and reverse modeling is performed to generate the high-precision three-dimensional model of the fractured bone of the patient using a computer.

Specifically, the steps of obtaining the DICOM data set of the patient by imaging techniques such as high-precision CT and performing reverse modeling to generate the high-precision three-dimensional model of the fractured bone of the patient using the computer are as follows.
a) Obtaining of DICOM data: the DICOM data set of the patient is obtained from a medical imaging device (such as CT scanning, and magnetic resonance imaging (MAI)). The DICOM data includes tomographic image information of the patient.
b) Preprocessing of DICOM data: the DICOM data is preprocessed, including noise removal, image calibration, and slice reconstruction. These preprocessing steps may ensure that high-quality image data is obtained.
c) Three-dimensional reconstruction: the DICOM slices are stacked using a three-dimensional reconstruction algorithm to generate a three-dimensional model of the bone of the patient. Common algorithms include voxel reconstruction, surface reconstruction, etc. These algorithms may convert slice images to consecutive three-dimensional bone surface or voxel representations.
d) Bone segmentation: bone segmentation is performed in the three-dimensional model, and the bone needing to be used is separated from other bones. The bone segmentation may be achieved using a manual segmentation or automatic segmentation algorithm.
e) Three-dimensional model repair and postprocessing: the bone model is repaired and postprocessed, including void filling, surface smoothing, anomaly structure removal, etc. These steps are conducive to generating a complete and accurate bone model.

In this embodiment, in the step of simulating intraoperative reduction on the high-precision three-dimensional model to obtain the post-reduction bone model 10, the three-dimensional model of the fractured bone is reduced using computer aided design (CAD) software or a specialized medical image processing tool. This includes recovering the fracture line to the normal anatomical position and ensuring the closure of the fracture line. For a patient with a fracture defect, if bone blocks for reverse modeling are incomplete, the integrity of the outer contour of the condyles of femur and that of the shaft of femur need to be guaranteed.

In this embodiment, in the step of establishing the post-reduction bone model 10 and performing finite element analysis to determine the force condition of the post-reduction bone model 10, a simplified force model, e.g., a force mode of the femur in a state of a 70 Kg adult standing on the ground with one foot when walking slowly, is used. The joint resultant force acting on the femur head is J=1588 N; and the force passes through the center of sphere of the femur head and forms an included angle of σ=24.4° with the line of force of the human body. The muscle force of the abducent muscle group is N=1039 N and forms an angle of θ=29.5° with the axis of the femur. The muscle force of the iliotibial band is R=169 N, and is vertically downward and parallel to the line of force of the human body. α=135°. Full fixation is applied to a position of the femur that is close to the knee joint. Forces for patients with other weights may be converted according to the forces for the patient with the standard weight.

The femoral cortical bone and the femoral cancellous bone are simplified as a continuous isotropic medium material. The internal fixation material has the elasticity modulus of 110 000 Mpa and the Poisson's ratio of 0.30. The normal cancellous bone has the elasticity modulus of 445 Mpa and the Poisson's ratio of 0.28.

After the finite element model is meshed, the maximum principal stress of each cell is calculated to find a stress concentration point. If the stress concentration point is on an outer side, a lateral plate is required for main force-bearing fixation. If the stress concentration point is on an inner side, a medial plate is required for main force-bearing fixation. Different geometrical features of a personalized plate may also be set according to the stress to be borne (e.g., if the stress is too high, the thickness or the width of the plate is increased to enhance the strength of the plate; and if the stress is too low, the width or the thickness of the plate may be appropriately reduced).

For example, if the body weight is 100 kg, the joint resultant force of the patient is J=1588*100/70=2268.6 N.

In this process, in the steps of extracting the point arrays of the cross section and the coronal plane from the medial femoral condyle part and the diaphysis part of the distal femur of the post-reduction bone model 10, connecting the point arrays of the cross section and the coronal plane to obtain the connection curve 13, and making optimization according to the anatomical condition to ensure that the shape and the structure of the entity part meet more accurate treatment requirements, and obtaining the solid body design of the plate body 11 according to the connection curve 13, intersection points of the coronal plane and the cross section reference plane with the bone model are the point arrays needing to be picked up. The point arrays of the cross section and the coronal plane are connected such that curves can be obtained by fitting. The curves of different planes are connected to form a sheet body, and optimization is made according to the anatomical condition to ensure that the shape and the structure after fitting meet the more accurate treatment requirements. Specific steps are as follows.

Generation of point arrays: a series of points in regions needing to be connected are selected on the high-precision three-dimensional model of the cross section and the coronal plane. These points should cover the whole connected region, and the anatomical structure and the fracture condition of the patient are taken into account. The density and positions of the points should be selected as needed.

Generation of connection curve 13: the selected points are used to generate the connection curve 13 by interpolation or other mathematical methods. These curves will connect the point arrays of different planes to form smooth transition. This step usually involves the CAD software or three-dimensional modeling tool.

Formation of connection curve 13 into sheet body: the connection curve 13 is scanned to form a sheet body. That is, an enclosed solid body is created around the connection curve 13. This may be achieved by extending the cross section of the curve along the curve path. This process will generate the basic shape of the sheet body.

Anatomical optimization: once the sheet body is generated, anatomical optimization may be made thereto. This includes fine adjusting the shape and the structure of the sheet body to ensure it meets the more accurate treatment requirements.

In this embodiment, the design method of an anatomical plate for treating a distal femoral fracture further includes: the post-reduction bone model (10) is combined with the anatomical plate (12) to obtain a combined model; finite element analysis is performed on the combined model; if the combined model meets a force requirement, it is determined that the design of the anatomical plate 12 is completed; and if the combined model does not meet the force requirement, the thickness and the shape of the plate body 11 are adjusted according to the force condition and the implantation condition of the post-reduction bone model 10 to obtain the anatomical plate 12 until the combined model meets the force requirement, thus guaranteeing that the anatomical plate 12 can meet the use requirement.

Specifically, the step of adjusting the thickness and the shape of the plate body 11 according to the force condition and the implantation condition of the post-reduction bone model 10 to obtain the anatomical plate 12 until the combined model meets the force requirement, thus guaranteeing that the anatomical plate 12 can meet the use requirement includes the following steps:

Establishment of finite element model after fracture reduction: the established finite element model is imported.

Establishment of finite element model of internal fixation system: the finite element model of the internal fixation system is added on the basis of the post-reduction model, including the plate and the screws. The same boundary condition and load condition with the original model are maintained.

Combination of internal fixation system and fracture model: the fracture model and the internal fixation system are assembled and combined in finite element analysis software. The correct position and direction of the internal fixator in the model are ensured.

Simulation of finite element analysis of internal fixation effect: the finite element analysis is performed to simulate the force condition of the fracture model under the action of the internal fixation system. Estimation of force condition of fractured part: the force distribution of the fractured region is determined, with particular focus on the force concentration point.

Calculation of force condition of sclerotin: the maximum stress received by the sclerotin is calculated according to the force condition applied by the internal fixation system. If the maximum stress of the sclerotin is less than 30 Mpa, it is regarded that the internal fixation effect is good, and the subsequent personalized design can be confirmed and carried forward.

Result verification and design adjustment: if the maximum stress of the sclerotin is greater than or equal to 30 Mpa, it is required to return to the design stage. Geometrical parameters (such as width and thickness) of the plate are adjusted to enhance the internal fixation effect. The finite element analysis of the internal fixation system is performed again to ensure that the internal fixation effect meets the requirement.

Final confirmation and design: when the internal fixation effect meets the expectation, the effectiveness of the personalized design is confirmed, and the final design confirmation is made. The detailed internal fixation scheme drawing or model is generated for use in the actual surgery.

The step of adjusting the thickness and the shape of the plate body 11 according to the force condition and the implantation condition of the post-reduction bone model 10 to obtain the anatomical plate 12 includes the following steps.

If the anatomical plate 12 is a medial plate and a non-force-bearing plate, it is determined that the anatomical plate 12 needs to be used in combination with a lateral plate, and a thickness of a distal end of the anatomical plate 12 is designed to be 3 mm to 5 mm, such as 3 mm, 4 mm, and 5 mm, and a thickness of a main body of the anatomical plate 12 to be 4 mm to 6 mm, such as 4 mm, 5 mm, and 6 mm.

If the anatomical plate 12 is a medial plate and a force-bearing plate, the thickness of the distal end of the anatomical plate 12 is adjusted according to a condition of soft tissue of the patient, and the thickness of the main body of the anatomical plate 12 is adjusted according to the force condition of the post-reduction bone model 10.

In this embodiment, if the medial plate is the non-force-bearing plate, it needs to be used in combination with the lateral plate. Since this plate is fixed in combination with the lateral plate, the thickness of the plate may be uniformly set such that the distal end is 2.5 mm thick in the vicinity of the joint surface and the main body is 4 mm thick.

If the medial plate is the force-bearing plate, the thickness of the distal end of the plate is adjusted according to different requirements of the soft tissue of the patient.

In this embodiment, the step of adjusting the thickness of the distal end of the anatomical plate 12 according to the condition of soft tissue of the patient includes the following steps.

If the thickness of the soft tissue of the patient is less than 20 mm, the thickness of the distal end of the anatomical plate 12 is set to be 3 mm to 3.5 mm, such as 3 mm, 3.2 mm, and 3.5 mm.

If the thickness of the soft tissue of the patient is 20 mm to 30 mm, the thickness of the distal end of the anatomical plate 12 is set to be 3.5 mm to 4.5 mm, such as 3.5 mm, 4 mm, and 4.5 mm.

If the thickness of the soft tissue of the patient is greater than 30 mm, the thickness of the distal end of the anatomical plate 12 is set to be 4.5 mm to 5 mm, such as 4.5 mm, 4.7 mm, and 5 mm.

In this embodiment, if the soft tissue is thin, it is easy to cause exposure and infection, and a thinner plate needs to be used, which is designed to be about 3 mm. If the soft tissue is normal, the standard thickness may be used, which is designed to be 3.5 mm. If the soft tissue is thick, it is allowed to use a thick plate, which is designed to be about 4 mm.

In this embodiment, the step of adjusting the thickness of the main body of the anatomical plate 12 according to the force condition of the post-reduction bone model 10 includes the following steps.

If the maximum stress of the post-reduction bone model 10 is less than 100 Mpa, the thickness of the main body of the anatomical plate 12 is set to be 4 mm to 4.5 mm, such as 4 mm, 4.2 mm, and 4.5 mm, to obtain the basic bending strength.

If the maximum stress of the post-reduction bone model 10 is 100 Mpa to 150 Mpa, the thickness of the main body of the anatomical plate 12 is set to be 4.5 mm to 5 mm, such as 4 mm, 4.5 mm, and 5 mm, to increase the bending strength.

If the maximum stress of the post-reduction bone model 10 is greater than 150 Mpa, the thickness of the main body of the anatomical plate 12 is set to be 5 mm to 6 mm, such as 5 mm, 5.5 mm, and 6 mm, for significantly improving the bending strength and ensuring the fixation effect.

The main body is the stress concentration region, the design needs to be optimized to prevent breakage. The thickness of the region is set according to the maximum stress result after the above-mentioned finite element analysis.

In this embodiment, the thickened body is cut from the front inner side according to the part needing to be fit and fixed so as to fix different bone fragments.

The step of adjusting the thickness and the shape of the plate body 11 according to the force condition and the implantation condition of the post-reduction bone model 10 to obtain the anatomical plate 12 further includes the following steps.

A lower edge of the plate body 11 is designed to be 1 mm to 3 mm (such as 1 mm, 2 mm, and 3 mm) higher than a femoral condyle joint surface, and a width of a distal end of plate body 11 is designed to be 25 mm to 35 mm, such as 25 mm, 30 mm, and 35 mm.

The distal end of the plate body 11 is designed to extend to a foremost edge of an inner side of the femoral condyle, and a main body of the plate body 11 is designed to extend to a front inner side of the distal femur. A width of the main body of the plate body 11 is designed to be 14 mm to 17 mm, such as 14 mm, 15 mm, 16 mm, and 17 mm.

In this embodiment, the distance of the lower edge of the plate to the joint surface of the femoral condyle needs to be about 2 mm, and the width of the farthest end is about 20 mm. The design needs to be made according to the outline of the femoral condyle of the patient. The distal end needs to be designed to extend to the foremost edge of the inner side of the femoral condyle. The main body needs to be set at the front inner side of the distal femur, and the circular arc is used in the middle for transition. The extended length of the main body needs to be determined according to the position of the fracture line. Generally, the width of the main body is set to be 15-17 mm. A length of 4 screw holes needs to be set outside the fracture line. The neck is a transition region, and its thickness and width are set for transition according to the design condition near the distal end.

In this embodiment, the step of adjusting the thickness and the shape of the plate body 11 according to the force condition and the implantation condition of the post-reduction bone model 10 to obtain the anatomical plate 12 further includes the following steps: a soft tissue condition around the bone of the patient is obtained, and a bevel angle and a fillet angle are added to the anatomical plate 12 in accordance with the soft tissue condition and an implantation position of the anatomical plate 12, thereby ensuring the optimization operation in the surgical process.

Specifically, the bevel angle design is added to the edge of the plate, and small bevel angles are adopted for transition at the joints of the front and rear ends with the flanges of the plate. This may avoid compression of the soft tissue by the plate angles, thereby reducing the wound tension and facilitating wound closure. The circular arc transition is added to the flange parts of the plate, and circular-arc-shaped smooth transition is provided for the contact surface of the plate and the bone. This may reduce the friction of the soft tissue caused by the edges of the plate, preventing tissue tearing after closure. The surface smoothness of the plate is optimized by using the precise molding process, and the surface roughness Ra is controlled to be less than 0.5 μm. The smooth surface may reduce the inflammatory response and the infection risk of the wound.

In this embodiment, the design method of an anatomical plate for treating a distal femoral fracture further includes the following steps: a position of a bone fragment is obtained and a position and a direction of a screw hole are determined in accordance with the position of the bone fragment and the force condition of the post-reduction bone model 10.

Specifically, the steps of obtaining the position of the bone fragment and determining the position and the direction of the screw hole in accordance with the position of the bone fragment and the force condition of the post-reduction bone model 10 include the following steps.

A plurality of first screw holes are designed in the distal end of the plate body 11, where the foremost first screw hole of the plurality of first screw holes is disposed corresponding to a lateral condyle, and the first screw hole of the plurality first screw holes that is located in the middle of farthest end is disposed corresponding to a medial condyle; and the other first screw holes are disposed perpendicular to a sagittal plane.

At least two screw holes are designed in the neck of the plate body 11, where the at least two screw holes are disposed perpendicular to the sagittal plane.

A third screw hole is designed in the main body of the plate body 11, where the third screw hole is perpendicular to the plate body 11.

In this embodiment, according to the conditions of the bone fragments and the mechanical performance results, the optimal positions of the screw holes are determined on the high-precision model and the direction of the screw holes is locked. The farthest end needs to be provided with five screws (passing through the first screw holes) in the vicinity of the joint surface. The direction of screws is designed according to the anatomic form of the patient. The foremost screw needs to be driven into the lateral condyle and the farthest middle screw needs to be driven in the direction of the medial condyle, thus ensuring the effective fixation of the condyles of both sides and increasing the implantation length. The other three screws for the distal end need to be driven perpendicularly to the sagittal plane of the patient, thus ensuring the maximum driving length. The neck is provided with two screws (passing through the second screw holes) for transition, and the two screws also needs to be driven perpendicularly to the sagittal plane of the patient. In order to enhance the stress dispersion effect, screws for the main body (passing through the third screw holes) are driven along the direction perpendicular to the setting surface of the plate. The screws for the distal end and the neck are all one-way fixation screws. The screws for the main body are arranged with reference to a conventional manner, and a composite screw hole is adopted. That is, a lag screw may be used, or a locking screw may also be used. The screw holes are provided at intervals of 10-15 mm.

The arrangement of the screw holes needs to follow the following principle: the positions of the bone fragments are identified according to CT, and points are selected on two sides of the fracture line for designing screw holes. The design of the screw holes needs to cover the bone fragments. However, the screw holes should not be too dense, thus avoiding influence on the intrabony blood supply. A “bamboo raft” type screw hole layout is designed for the platform near the joint. The screw holes are in a mesh layout. The holes are arranged at intervals of 10-15 mm, and should not be too dense or too sparse. The fixation of the front and rear cortical layers of the diaphysis shall be taken into account in screw setting. The fixation of the front and rear cortical layers of the diaphysis can be realized.

Another embodiment of the present disclosure provides an anatomical plate 12. The anatomical plate 12 is manufactured in accordance with the design method of an anatomical plate for treating a distal femoral fracture described above. Therefore, the anatomical plate 12 can match the anatomic form or the fracture form of a patient, and thus can provide enough stability, leading to stable fixation and being conducive to the healing process of the fracture.

Specifically, the shape and the size of the anatomical plate 12 may be customized according to the fracture condition and the anatomical structure of a specific patient. The design of the anatomical plate 12 takes the mechanical conduction condition and the fixation condition of a plurality of bone fragments into full account to provide three-dimensional positioning stability. For the length and the shape of the anatomical plate 12, the requirement of the operative route, the anatomical attachment condition, and postoperative suture are taken into full consideration. The design of the anatomical plate 12 takes the biomechanical requirement of fracture healing into full consideration to facilitate faster healing and the rehabilitation of the patient.

It should be noted that the terminology used herein is for the purpose of describing particular embodiments only and is not intended to limit exemplary embodiments according to the present disclosure. As used herein, unless otherwise specified herein, the singular forms are also intended to include the plural forms. In addition, it should also be understood that when the terms “comprise” and/or “include” are used in this specification, they specify the presence of features, steps, operations, devices, components, and/or combinations thereof.

Unless otherwise specified, the relative arrangement, numerical expressions and numerical values of components and steps set forth in these examples do not limit the scope of the present disclosure. Meanwhile, it should be understood that for ease of description, each portion in the drawings is not necessarily drawn to the actual scale. The technologies, methods, and devices known to those of ordinary skill in the art may not be discussed in detail, but where appropriate, the technologies, methods, and devices should be regarded as part of the specification. In all examples shown and discussed herein, any specific value should be interpreted as merely exemplary, rather than restrictive. Therefore, other examples of the exemplary embodiments may have different values. It should be noted that similar reference signs and letters represent similar items in the accompanying drawings below. Therefore, once an item is defined in one drawing, it does not need to be further discussed in subsequent drawings.

It should be understood that, in the description of the present disclosure, terms such as “front”, “rear”, “upper”, “lower”, “left”, “right”, “transverse”, “longitudinal”, “vertical”, “horizontal”, “top” and “bottom” indicate orientation or position relationships based on the accompanying drawings. Unless otherwise specified, these terms are merely intended to facilitate or simplify the description of the present disclosure, rather than to indicate or imply that the mentioned device or components must have a specific orientation and must be constructed and operated in a specific orientation. Therefore, they should not be construed as a limitation to the protection scope of the present disclosure. The orientation terms “inner” and “outer” refer to the inner and outer parts relative to the contour of the mentioned component.

For ease of description, spatially relative terms, such as “above”, “on the upper side of”, “on the upper surface of” and “on”, can be used to describe the spatial positional relationship between components or features shown in the figure. It should be understood that the spatially relative terms are intended to encompass different orientations of the components in use or operation in addition to those shown in the figure. For example, if a component in the figure is inverted, it is described as a component “above other component or structure” or “on other component or structure”. Therefore, the component will be positioned as “below other component or structure” or “under other component or structure”. Therefore, the exemplary term “above” may include both orientations “above” and “below”. The component may also be positioned in other different ways (rotated by 90 degrees or in other orientations), but the relative description of the space should be explained accordingly.

In addition, it needs to be noted that the use of such words as “first” and “second” to define components is merely intended to distinguish the corresponding components. Unless otherwise stated, such words have no special meaning and thus cannot be construed as limiting the protection scope of the present disclosure.

The foregoing are merely descriptions of the preferred embodiments of the present disclosure and not intended to limit the present disclosure, and various changes and modifications of the present disclosure may be made by those skilled in the art. Any modifications, equivalent substitutions, improvements and the like made within the spirit and principle of the present disclosure should be included within the protection scope of the present disclosure.

1. A design method of an anatomical plate for treating a distal femoral fracture, comprising:
obtaining a high-precision three-dimensional model of a fractured bone of a patient;
simulating intraoperative reduction on the high-precision three-dimensional model to obtain a post-reduction bone model (10);
establishing a finite element model according to the post-reduction bone model (10) and performing finite element analysis to determine a force condition of the post-reduction bone model (10);
extracting point arrays of a cross section and a coronal plane from a medial femoral condyle part and a diaphysis part of a distal femur of the post-reduction bone model (10), connecting the point arrays of the cross section and the coronal plane to obtain a connection curve (13), and obtaining a solid body design of a plate body (11) according to the connection curve (13); and
adjusting a thickness and a shape of the plate body (11) according to the force condition and an implantation condition of the post-reduction bone model (10) to obtain an anatomical plate (12).

2. The design method of an anatomical plate for treating a distal femoral fracture according to claim 1, further comprising:
combining the post-reduction bone model (10) with the anatomical plate (12) to obtain a combined model;
performing finite element analysis on the combined model; if the combined model meets a force requirement, determining that the design of the anatomical plate (12) is completed; and if the combined model does not meet the force requirement, adjusting the thickness and the shape of the plate body (11) according to the force condition and the implantation condition of the post-reduction bone model (10) to obtain the anatomical plate (12) until the combined model meets the force requirement.

3. The design method of an anatomical plate for treating a distal femoral fracture according to claim 1, wherein the adjusting a thickness and a shape of the plate body (11) according to the force condition and an implantation condition of the post-reduction bone model (10) to obtain an anatomical plate (12) comprises:
if the anatomical plate (12) is a medial plate and a non-force-bearing plate, determining that the anatomical plate (12) needs to be used in combination with a lateral plate, and designing a thickness of a distal end of the anatomical plate (12) to be 3 mm to 5 mm and a thickness of a main body of the anatomical plate (12) to be 4 mm to 6 mm; and
if the anatomical plate (12) is a medial plate and a force-bearing plate, adjusting the thickness of the distal end of the anatomical plate (12) according to a condition of soft tissue of the patient, and adjusting the thickness of the main body of the anatomical plate (12) according to the force condition of the post-reduction bone model (10).

4. The design method of an anatomical plate for treating a distal femoral fracture according to claim 3, wherein the adjusting the thickness of the distal end of the anatomical plate (12) according to a condition of soft tissue of the patient comprises:
if a thickness of the soft tissue of the patient is less than 20 mm, setting the thickness of the distal end of the anatomical plate (12) to be 3 mm to 3.5 mm;
if the thickness of the soft tissue of the patient is 20 mm to 30 mm, setting the thickness of the distal end of the anatomical plate (12) to be 3.5 mm to 4.5 mm; and
if the thickness of the soft tissue of the patient is greater than 30 mm, setting the thickness of the distal end of the anatomical plate (12) to be 4.5 mm to 5 mm.

5. The design method of an anatomical plate for treating a distal femoral fracture according to claim 3, wherein the adjusting the thickness of the main body of the anatomical plate (12) according to the force condition of the post-reduction bone model (10) comprises:
if a maximum stress of the post-reduction bone model (10) is less than 100 Mpa, setting the thickness of the main body of the anatomical plate (12) to be 4 mm to 4.5 mm;
if the maximum stress of the post-reduction bone model (10) is 100 Mpa to 150 Mpa, setting the thickness of the main body of the anatomical plate (12) to be 4.5 mm to 5 mm; and
if the maximum stress of the post-reduction bone model (10) is greater than 150 Mpa, setting the thickness of the main body of the anatomical plate (12) to be 5 mm to 6 mm.

6. The design method of an anatomical plate for treating a distal femoral fracture according to claim 1, wherein the adjusting a thickness and a shape of the plate body (11) according to the force condition and an implantation condition of the post-reduction bone model (10) to obtain an anatomical plate (12) further comprises:
designing a lower edge of the plate body (11) to be 1 mm to 3 mm higher than a femoral condyle joint surface, and designing a width of a distal end of the plate body (11) to be 25 mm to 35 mm;
designing the distal end of the plate body (11) to extend to a foremost edge of an inner side of the femoral condyle, and designing a main body of the plate body (11) to extend to a front inner side of the distal femur; and
designing a width of the main body of the plate body (11) to be 14 mm to 17 mm.

7. The design method of an anatomical plate for treating a distal femoral fracture according to claim 1, wherein the adjusting a thickness and a shape of the plate body (11) according to the force condition and an implantation condition of the post-reduction bone model (10) to obtain an anatomical plate (12) further comprises:
obtaining a soft tissue condition around the bone of the patient, and adding a bevel angle and a fillet angle to the anatomical plate (12) in accordance with the soft tissue condition and an implantation position of the anatomical plate (12).

8. The design method of an anatomical plate for treating a distal femoral fracture according to claim 1, further comprising:
obtaining a position of a bone fragment and determining a position and a direction of a screw hole in accordance with the position of the bone fragment and the force condition of the post-reduction bone model (10).

9. The design method of an anatomical plate for treating a distal femoral fracture according to claim 8, wherein the obtaining a position of a bone fragment and determining a position and a direction of a screw hole in accordance with the position of the bone fragment and the force condition of the post-reduction bone model (10) comprise:
designing a plurality of first screw holes in the distal end of the plate body (11), wherein the foremost first screw hole of the plurality of first screw holes is disposed corresponding to a lateral condyle, and the first screw hole of the plurality of first screw holes that is located in the middle of a farthest end is disposed corresponding to a medial condyle; and the other first screw holes are disposed perpendicular to a sagittal plane;
designing at least two screw holes in a neck of the plate body (11), wherein the at least two screw holes are disposed perpendicular to the sagittal plane; and
designing a third screw hole in the main body of the plate body (11), wherein the third screw hole is perpendicular to the plate body (11).

10. An anatomical plate, manufactured in accordance with the design method of an anatomical plate for treating a distal femoral fracture according to claim 1.

11. The anatomical plate according to claim 10, further comprising:
combining the post-reduction bone model (10) with the anatomical plate (12) to obtain a combined model;
performing finite element analysis on the combined model; if the combined model meets a force requirement, determining that the design of the anatomical plate (12) is completed; and if the combined model does not meet the force requirement, adjusting the thickness and the shape of the plate body (11) according to the force condition and the implantation condition of the post-reduction bone model (10) to obtain the anatomical plate (12) until the combined model meets the force requirement.

12. The anatomical plate according to claim 10, wherein the adjusting a thickness and a shape of the plate body (11) according to the force condition and an implantation condition of the post-reduction bone model (10) to obtain an anatomical plate (12) comprises:
if the anatomical plate (12) is a medial plate and a non-force-bearing plate, determining that the anatomical plate (12) needs to be used in combination with a lateral plate, and designing a thickness of a distal end of the anatomical plate (12) to be 3 mm to 5 mm and a thickness of a main body of the anatomical plate (12) to be 4 mm to 6 mm; and
if the anatomical plate (12) is a medial plate and a force-bearing plate, adjusting the thickness of the distal end of the anatomical plate (12) according to a condition of soft tissue of the patient, and adjusting the thickness of the main body of the anatomical plate (12) according to the force condition of the post-reduction bone model (10).

13. The anatomical plate according to claim 12, wherein the adjusting the thickness of the distal end of the anatomical plate (12) according to a condition of soft tissue of the patient comprises:
if a thickness of the soft tissue of the patient is less than 20 mm, setting the thickness of the distal end of the anatomical plate (12) to be 3 mm to 3.5 mm;
if the thickness of the soft tissue of the patient is 20 mm to 30 mm, setting the thickness of the distal end of the anatomical plate (12) to be 3.5 mm to 4.5 mm; and
if the thickness of the soft tissue of the patient is greater than 30 mm, setting the thickness of the distal end of the anatomical plate (12) to be 4.5 mm to 5 mm.

14. The anatomical plate according to claim 12, wherein the adjusting the thickness of the main body of the anatomical plate (12) according to the force condition of the post-reduction bone model (10) comprises:
if a maximum stress of the post-reduction bone model (10) is less than 100 Mpa, setting the thickness of the main body of the anatomical plate (12) to be 4 mm to 4.5 mm;
if the maximum stress of the post-reduction bone model (10) is 100 Mpa to 150 Mpa, setting the thickness of the main body of the anatomical plate (12) to be 4.5 mm to 5 mm; and
if the maximum stress of the post-reduction bone model (10) is greater than 150 Mpa, setting the thickness of the main body of the anatomical plate (12) to be 5 mm to 6 mm.

15. The anatomical plate according to claim 10, wherein the adjusting a thickness and a shape of the plate body (11) according to the force condition and an implantation condition of the post-reduction bone model (10) to obtain an anatomical plate (12) further comprises:
designing a lower edge of the plate body (11) to be 1 mm to 3 mm higher than a femoral condyle joint surface, and designing a width of a distal end of the plate body (11) to be 25 mm to 35 mm;
designing the distal end of the plate body (11) to extend to a foremost edge of an inner side of the femoral condyle, and designing a main body of the plate body (11) to extend to a front inner side of the distal femur; and
designing a width of the main body of the plate body (11) to be 14 mm to 17 mm.

16. The anatomical plate according to claim 10, wherein the adjusting a thickness and a shape of the plate body (11) according to the force condition and an implantation condition of the post-reduction bone model (10) to obtain an anatomical plate (12) further comprises:
obtaining a soft tissue condition around the bone of the patient, and adding a bevel angle and a fillet angle to the anatomical plate (12) in accordance with the soft tissue condition and an implantation position of the anatomical plate (12).

17. The anatomical plate according to claim 10, further comprising:
obtaining a position of a bone fragment and determining a position and a direction of a screw hole in accordance with the position of the bone fragment and the force condition of the post-reduction bone model (10).

18. The anatomical plate according to claim 17, wherein the obtaining a position of a bone fragment and determining a position and a direction of a screw hole in accordance with the position of the bone fragment and the force condition of the post-reduction bone model (10) comprise:
designing a plurality of first screw holes in the distal end of the plate body (11), wherein the foremost first screw hole of the plurality of first screw holes is disposed corresponding to a lateral condyle, and the first screw hole of the plurality of first screw holes that is located in the middle of a farthest end is disposed corresponding to a medial condyle; and the other first screw holes are disposed perpendicular to a sagittal plane;
designing at least two screw holes in a neck of the plate body (11), wherein the at least two screw holes are disposed perpendicular to the sagittal plane; and
designing a third screw hole in the main body of the plate body (11), wherein the third screw hole is perpendicular to the plate body (11).
